(19) United States
(12) Patent Application Publication
(10) Pub. No.: US 2007/0287855 A1
(43) Pub. Date: Dec. 13, 2007

(54) Process for Preparing Fluorinated Molecules

(75) Inventors: Thomas Geller, Odenthal (DE); Norbert Lui, Odenthal (DE)

Correspondence Address: FROMMER LAWRENCE & HAUG, 745 FIFTH AVENUE- 10TH FL., NEW YORK, NY, 10151, US

(73) Assignee: Bayer CropScience AG, Frankfurt (DE)

(21) Appl. No.: 11/758,799

(22) Filed: Jun. 6, 2007

(30) Foreign Application Priority Data
Jun. 8, 2006 (DE) 10 2006 027 089.4

(51) Int. Cl. C07C 69/76; C07C 69/63

(52) U.S. Cl. 560/83; 560/227

(57) ABSTRACT

The present invention relates to a process for preparing α-fluorinated esters from α-hydroxy esters by reaction with a dihalocarbonyl compound (or an equivalent) to give haloformates and further to give fluoroformates, which are then decomposed thermally in the presence of suitable catalysts. The invention further relates to the individual steps of the process and in some cases to novel fluoroformates.

This application claims benefit under 35 U.S.C. 119(a) of German patent application DE 10 2006 027 089.4, filed on 8 Jun. 2006.

Any foregoing applications, including German patent application DE 10 2006 027 089.4, and all documents cited therein or during their prosecution (“application cited documents”) and all documents cited or referenced in the application cited documents, and all documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention.

The invention relates to the technical field of chemical processes for preparing fluorine compounds, more specifically of processes for preparing carboxylic esters which contain a fluorine atom as a substituent in the α-position (alpha).

It is already known that esters which, in the α-position (alpha), contain a fluorine atom as a substituent can be prepared by the reaction of α-hydroxy esters with thionyl chloride to give the chlorosulfite, reaction of the chlorosulfite with a fluoride source to give the fluorosulfite and subsequent thermal decomposition, if appropriate with amine or pyridine catalysis (DE 1122505; GB 899127); see scheme 1:

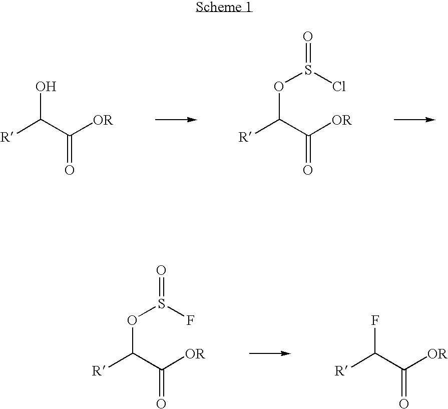

The use of chiral, non-racemic α-hydroxy esters allows this reaction also to be used in an otherwise identical reaction in order to prepare the target products as chiral, non-racemic compounds (WO-A-2006/037887, FR-A1-2876100). The chlorosulfites of the α-hydroxy esters which are required in these processes can be prepared by known processes (for example EP-A-0056981, DE-A1-3102516, U.S. Pat. No. 4,408,068).

A disadvantage in this method of preparation is the formation of SO₂ as an offgas and the decomposition, which is observed as a side reaction, of the chlorosulfites to the corresponding α-chlorinated esters (for example DE-A-3102516, utilized there as the main reaction).

Owing to very similar properties, the chlorinated esters can be separated from the target product only with difficulty.

It is likewise known that α-fluorinated esters can also be prepared by converting α-hydroxy esters to the corresponding sulfonic esters and then reacting the sulfonic ester with a fluoride source (for example potassium fluoride) (for example DE-A-4131242). This reaction can be utilized enantioselectively to prepare chiral, non-racemic target products when the reactant is used in chiral, non-racemic form (see scheme 2 using the example of an enantiomeric α-fluorinated ester).

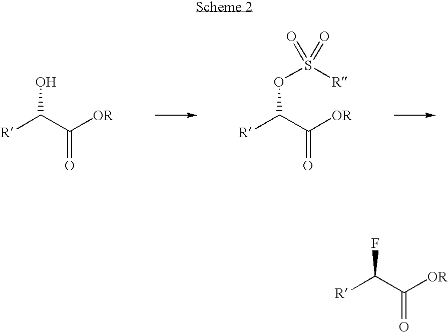

Caused by the high molecular weight of the leaving group, it becomes necessary to manage relatively large masses during the process in order to obtain a relatively light target product at the end. As a result of this and as a result of the poor biodegradability of the leaving groups, there are problems on the industrial scale.

DE-A-3836855 discloses that α-fluorinated esters can be obtained by deaminating an amino acid with sodium nitrite in the presence of hydrogen fluoride and pyridine. Moreover, DE-A-3836855 describes the ring-opening of an epoxide with a mixture of HF and pyridine, which, after oxidation and esterification, likewise leads to α-fluorinated esters. In both processes, the amounts of hydrogen fluoride and pyridine used are relatively high, which complicates their industrial usability. Furthermore, the deamination leads, in the case of the preparation of chiral, non-racemic products, only to unsatisfactory enantiomeric excesses (less than 60%).

Furthermore, Tetrahedron Lett., 2002, 43, 4275-4279 discloses that alkyl fluoroformates can be decomposed thermally to fluoroalkanes in the presence of hexabutylguanidinium fluoride (see scheme 3), the reaction proceeding largely enantioselectively. The alkyl fluoroformates used can be prepared according to J. Org. Chem. 1956, 21, 1319-1320 or Tetrahedron Lett., 2002, 43, 4275-4279 by reaction of hydroxyalkanes with fluorobromophosgene, fluorochlorophosgene, difluorophosgene or potassium fluoride+UV radiation.

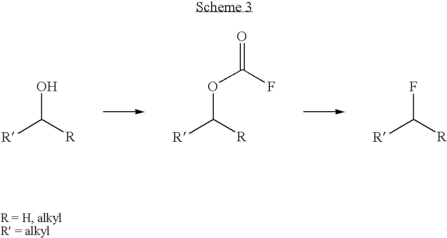

Since α-fluorinated esters, especially in their chiral, non-racemic form, constitute important intermediates for biologically active compounds, it is desirable to find a preparation route which makes the fluorinated esters preparable industrially in a simple manner.

The invention provides a process for preparing compounds of the formula (IV), optionally in optically active form,

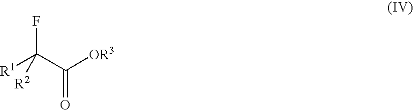

in which
R¹ is hydrogen, (C₁-C₂₄)-alkyl, (C₂-C₂₄)-alkenyl, (C₂-C₂₄)-alkynyl, (C₃-C₉)-cycloalkyl, (C₁-C₆)-alkyl-(C₃-C₉)-cycloalkyl or a radical of the formula —CO₂R⁴, —(CH₂)ₙCO₂R⁴, —COR⁴, —SOR⁴ or —SO₂R⁴, where n is an integer from 0 to 12, or aryl, heterocyclyl, phenyl-(C₁-C₆)-alkyl or heterocyclyl-(C₁-C₆)-alkyl, where each of the 4 latter radicals, on the ring, is unsubstituted or substituted by one or more radicals from the group of halogen, (C₁-C₁₂)-alkyl, (C₁-C₁₂)-alkoxy, (C₁-C₁₂)-haloalkyl and (C₁-C₁₂)-haloalkoxy, and heterocyclyl is in each case a heterocyclic radical having 1 to 3 heteroatoms from the group of N, O and S,
R² is hydrogen, (C₁-C₂₄)-alkyl, (C₂-C₂₄)-alkenyl, (C₂-C₂₄)-alkynyl, (C₃-C₉)-cycloalkyl, (C₁-C₆)-alkyl-(C₃-C₉)-cycloalkyl or a radical of the formula —CO₂R⁴, —(CH₂)ₙCO₂R⁴, —COR⁴, —SOR⁴ or —SO₂R⁴, where n is an integer from 0 to 12, or optionally substituted aryl, preferably phenyl, which is unsubstituted or substituted by one or more radicals from the group of halogen, (C₁-C₁₂)-alkyl, (C₁-C₁₂)-alkoxy, (C₁-C₁₂)-haloalkyl and (C₁-C₁₂)-haloalkoxy,
R³ is (C₁-C₂₄)-alkyl, (C₂-C₂₄)-alkenyl, (C₂-C₂₄)-alkynyl, (C₃-C₉)-cycloalkyl, (C₁-C₆)-alkyl-(C₃-C₉)-cycloalkyl, aryl, heterocyclyl, phenyl-(C₁-C₆)-alkyl or heterocyclyl-(C₁-C₆)-alkyl, where each of the 4 latter radicals, on the ring, is unsubstituted or substituted by one or more radicals from the group of halogen, (C₁-C₁₂)-alkyl, (C₁-C₁₂)-alkoxy, (C₁-C₁₂)-haloalkyl and (C₁-C₁₂)-haloalkoxy, and heterocyclyl is in each case a heterocyclic radical having 1 to 3 heteroatoms from the group of N, O and S,
R⁴ is (C₁-C₂₄)-alkyl, (C₂-C₂₄)-alkenyl, (C₂-C₂₄)-alkynyl, (C₃-C₉)-cycloalkyl, (C₁-C₆)-alkyl-(C₃-C₉)-cycloalkyl, aryl, heterocyclyl, phenyl-(C₁-C₆)-alkyl or heterocyclyl-(C₁-C₆)-alkyl, where each of the 4 latter radicals, on the ring, is unsubstituted or substituted by one or more radicals from the group of halogen, (C₁-C₁₂)-alkyl, (C₁-C₁₂)-alkoxy, (C₁-C₁₂)-haloalkyl and (C₁-C₁₂)-haloalkoxy, and heterocyclyl is in each case a heterocyclic radical having 1 to 3 heteroatoms from the group of N, O and S,
   which comprises
(a) reacting a compound of the formula (I)

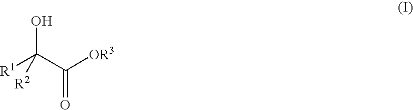

in which R¹, R² and R³ are each as defined in formula (IV)
with a dihalocarbonyl compound or an equivalent thereof to give the compound of the formula (II) [variant (a1)] or (III) [variant (a2)]

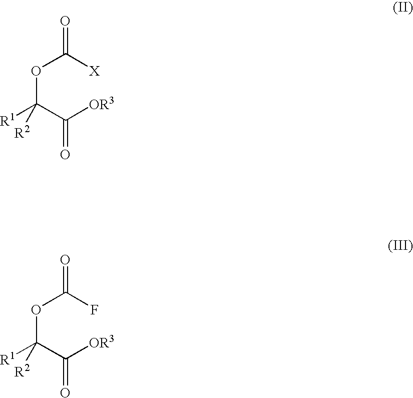

where, in the formulae (II) and (III),
R¹, R² and R³ are each as defined in formula (IV) and
X is a halogen atom from the group of Cl or Br,
and
(b) in the case that the compound (II) has been obtained in stage (a) according to variant (a1), reacting the compound (II) with a fluorinating reagent to give the compound of the formula (III) mentioned,
   (c) reacting the compound of the formula (III) obtained in stage (a) or (b) thermally, optionally in the presence of a catalyst, with decarboxylation to give the compound of the formula (IV) mentioned.

In the formula (I) and all formulae which follow, the alkyl radicals, including in the composite definitions such as alkoxy, haloalkyl and haloalkoxy and also the corresponding unsaturated and/or substituted radicals, may each be straight-chain or branched in the carbon skeleton.

The expression “(C₁-C₄)alkyl” is a brief notation for alkyl having from one to 4 carbon atoms, i.e. encompasses the methyl, ethyl, 1-propyl, 2-propyl, 1-butyl, 2-butyl, 2-methylpropyl or tert-butyl radicals. General alkyl radicals with a larger specified number of carbon atoms, for example “(C₁-C₆)alkyl” correspondingly also include straight-chain or branched alkyl radicals having a larger number of carbon atoms, i.e., according to the example, also the alkyl radicals having 5 and 6 carbon atoms. Unless stated specifically, preference is given to the lower carbon skeletons, for example having from 1 to 6 carbon atoms, or having from 2 to 6 carbon atoms in the case of unsaturated groups, in the case of the hydrocarbon radicals such as alkyl, alkenyl and alkynyl radicals, including in combined radicals. Alkyl radicals, including in the combined definitions such as alkoxy, haloalkyl, etc., are, for example, methyl, ethyl, n- or i-propyl, n-, i-, t- or 2-butyl, pentyls, hexyls such as n-hexyl, i-hexyl and 1,3-dimethylbutyl, heptyls such as n-heptyl, 1-methylhexyl and 1,4-dimethylpentyl; alkenyl and alkynyl radicals are defined as the possible unsaturated radicals corresponding to the alkyl radicals; alkenyl is, for example, vinyl, allyl, 1-methyl-2-propenyl, 2-methyl-2-propenyl, 2-butenyl, pentenyl, 2-methylpentenyl or hexenyl group, preferably allyl, 1-methylprop-2-en-1-yl, 2-methylprop-2-en-1-yl, but-2-en-1-yl, but-3-en-1-yl, 1-methylbut-3-en-1-yl or 1-methylbut-2-en-1-yl.

Alkenyl also includes in particular straight-chain or branched hydrocarbon radicals having more than one double bond, such as 1,3-butadienyl and 1,4-pentadienyl, but also allenyl or cumulenyl radicals having one or more cumulated double bonds, for example allenyl (1,2-propadienyl), 1,2-butadienyl and 1,2,3-pentatrienyl.

Alkynyl is, for example, propargyl, but-2-yn-1-yl, but-3-yn-1-yl, 1-methylbut-3-yn-1-yl. Alkynyl also includes, in particular, straight-chain or branched hydrocarbon radicals having more than one triple bond or else having one or more triple bonds and one or more double bonds, for example 1,3-butatrienyl or 3-penten-1-yn-1-yl.

Alkylidene, for example also in the form of (C₁-C₁₀)alkylidene, is the radical of a straight-chain or branched alkane which is bonded via a double bond, the position of the binding site not being fixed. In the case of a branched alkane, of course, only positions at which two hydrogen atoms may be replaced by the double bond are possible; radicals are, for example, ═CH₂, ═CH—CH₃, ═C(CH₃)—CH₃, ═C(CH₃)—C₂H₅ or ═C(C₂H₅)—C₂H₅.

Cycloalkyl is a carbocyclic saturated ring system having preferably 3-8 carbon atoms, for example cyclopropyl, cyclobutyl, cyclopentyl or cyclohexyl. In the case of substituted cycloalkyl, cyclic systems with substituents are included, where the substituents are bonded by a double bond on the cycloalkyl radical, for example an alkylidene group such as methylidene. In the case of substituted cycloalkyl, polycyclic aliphatic systems are also included, for example bicyclo[1.1.0]butan-1-yl, bicyclo[1.1.0]butan-2-yl, bicyclo[2.1.0]pentan-1-yl, bicyclo[2.1.0]pentan-2-yl, bicyclo[2.1.0]pentan-5-yl, adamantan-1-yl and adamantan-2-yl.

Halogen is, for example, fluorine, chlorine, bromine or iodine. Haloalkyl, -alkenyl and -alkynyl are, respectively, alkyl, alkenyl and alkynyl substituted partly or fully by identical or different halogen atoms, preferably from the group of fluorine, chlorine and bromine, in particular from the group of fluorine and chlorine, for example monohaloalkyl, perhaloalkyl, CF₃, CHF₂, CH₂F, CF₃CF₂, CH₂FCHCl, CCl₃, CHCl₂, CH₂CH₂Cl; haloalkoxy is, for example OCF₃, OCHF₂, OCH₂F, CF₃CF₂O, OCH₂CF₃ and OCH₂CH₂Cl; the same applies to haloalkenyl and other halogen-substituted radicals.

Aryl is a mono-, bi- or polycyclic aromatic system, for example phenyl, naphthyl, tetrahydronaphthyl, indenyl, indanyl, pentalenyl, fluorenyl and the like, preferably phenyl.

A heterocyclic radical or ring (heterocyclyl) can be saturated, unsaturated or heteroaromatic; unless defined otherwise, it preferably contains one or more, in particular 1, 2 or 3, heteroatoms in the heterocyclic ring, preferably from the group of N, O and S; it is preferably an aliphatic heterocyclyl radical having from 3 to 7 ring atoms or a heteroaromatic radical having 5 or 6 ring atoms. The heterocyclic radical may, for example, be a heteroaromatic radical or ring (heteroaryl), for example a mono-, bi- or polycyclic aromatic system in which at least 1 ring contains one or more heteroatoms. It is preferably a heteroaromatic ring having a heteroatom from the group of N, O and S, for example pyridyl, pyrrolyl, thienyl or furyl; it is also preferably a corresponding heteroaromatic ring having 2 or 3 heteroatoms, for example pyrimidinyl, pyridazinyl, pyrazinyl, triazinyl, thiazolyl, thiadiazolyl, oxazolyl, isoxazolyl, pyrazolyl, imidazolyl and triazolyl. It is also preferably a partially or fully hydrogenated heterocyclic radical having one heteroatom from the group of N, O and S, for example oxiranyl, oxetanyl, oxolanyl (=tetrahydrofuryl), oxanyl, pyrrolinyl, pyrrolidyl or piperidyl.

It is also preferably a partially or fully hydrogenated heterocyclic radical having 2 heteroatoms from the group of N, O and S, for example piperazinyl, dioxolanyl, oxazolinyl, isoxazolinyl, oxazolidinyl, isoxazolidinyl and morpholinyl.

Possible substituents for a substituted heterocyclic radical include the substituents specified below, and additionally also oxo. The oxo group may also occur on the ring heteroatoms which may exist in various oxidation states, for example in the case of N and S.

Preferred examples of heterocyclyl are a heterocyclic radical having from 3 to 6 ring atoms from the group of pyridyl, thienyl, furyl, pyrrolyl, oxiranyl, 2-oxetanyl, 3-oxetanyl, oxolanyl (=tetrahydrofuryl), pyrrolidyl, piperidyl, especially oxiranyl, 2-oxetanyl, 3-oxetanyl or oxolanyl, or a heterocyclic radical having two or three heteroatoms, for example pyrimidinyl, pyridazinyl, pyrazinyl, triazinyl, thienyl, thiazolyl, thiadiazolyl, oxazolyl, isoxazolyl, pyrazolyl, triazolyl, piperazinyl, dioxolanyl, oxazolinyl, isoxazolinyl, oxazolidinyl, isoxazolidinyl or morpholinyl.

When a base structure is substituted “by one or more radicals” from a list of radicals (=group) or a generically defined group of radicals, this in each case includes simultaneous substitution by a plurality of identical and/or structurally different radicals.

Substituted radicals, such as a substituted aryl, phenyl, heterocyclyl and heteroaryl radical, are, for example, a substituted radical derived from the unsubstituted base structure, where the substituents are, for example, one or more, preferably 1, 2 or 3, radicals from the group of halogen, alkoxy, alkylthio, hydroxyl, amino, nitro, carboxyl, cyano, azido, alkoxycarbonyl, alkylcarbonyl, formyl, carbamoyl, mono- and dialkylaminocarbonyl, substituted amino such as acylamino, mono- and dialkylamino, and alkylsulfinyl, alkylsulfonyl alkyl, haloalkyl, alkylthioalkyl, alkoxyalkyl, optionally substituted mono- and dialkylaminoalkyl and hydroxyalkyl; in the term “substituted radicals”, such as substituted alkyl, etc., substituents include, in addition to the saturated hydrocarbon radicals mentioned, corresponding unsaturated aliphatic and aromatic radicals, such as optionally substituted alkenyl, alkynyl, alkenyloxy, alkynyloxy, phenyl, phenoxy, etc.

The substituents mentioned by way of example (“first substituent level”) may, when they contain hydrocarbon moieties, optionally be further substituted there (“second substituent level”), for example by one of the substituents as defined for the first substituent level. Corresponding further substituent levels are possible. The term “substituted radical” preferably includes only one or two substituent levels.

Preferred substituents for the substituent levels are, for example:

In the case of radicals with carbon atoms, preference is given to those having from 1 to 6 carbon atoms, preferably from 1 to 4 carbon atoms, in particular 1 or 2 carbon atoms. In general, preferred substituents are those from the group of halogen, e.g. fluorine and chlorine, (C₁-C₄)alkyl, preferably methyl or ethyl, (C₁-C₄)haloalkyl, preferably trifluoromethyl, (C₁-C₄)alkoxy, preferably methoxy or ethoxy, (C₁-C₄)haloalkoxy, nitro and cyano. Particular preference is given to the substituents methyl, methoxy, fluorine and chlorine.

Optionally substituted phenyl is preferably phenyl which is unsubstituted or mono- or polysubstituted, preferably up to trisubstituted, by identical or different radicals from the group of halogen, (C₁-C₄)alkyl, (C₁-C₄)alkoxy, (C₁-C₄)haloalkyl, (C₁-C₄)haloalkoxy and nitro, for example o-, m- and p-tolyl, dimethylphenyls, 2-, 3- and 4-chlorophenyl, 2-, 3- and 4-trifluoro- and -trichlorophenyl, 2,4-, 3,5-, 2,5- and 2,3-dichlorophenyl, o-, m- and p-methoxyphenyl.

The formula (I) and formulae which follow also include all stereoisomers and mixtures thereof. Such compounds of the formula (I) contain one or more asymmetric carbon atoms or else double bonds which are not specified separately in the formula (I). The possible stereoisomers defined by their specific three-dimensional shape, such as enantiomers, diastereomers, Z- and E-isomers, are all encompassed by the formula (I) and can, in the preferred enantioselective procedure, be prepared selectively when optically active starting materials are used.

It is noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the invention.

It is further noted that the invention does not intend to encompass within the scope of the invention any previously disclosed product, process of making the product or method of using the product, which meets the written description and enablement requirements of the USPTO (35 U.S.C. 112, first paragraph) or the EPO (Article 83 of the EPC), such that applicant(s) reserve the right and hereby disclose a disclaimer of any previously described product, method of making the product or process of using the product.

Preferred chain lengths for alkyl, alkenyl, alkynyl in the R¹, R², R³ and R⁴ radicals are C₁-C₁₂, more preferably C₁-C₆, very particularly C₁-C₄.

The preferred ring size for cycloalkyl in R¹, R², R³ and R⁴ is C₃-C₇, especially C₃-C₆.

Preferably,
R¹ is hydrogen, (C₁-C₁₂)-alkyl, (C₂-C₁₂)-alkenyl, (C₂-C₁₂)-alkynyl, (C₃-C₆)-cycloalkyl, (C₁-C₄)-alkyl-(C₃-C₆)-cycloalkyl or a radical of the formula —CO₂R⁴, —(CH₂)ₙCO₂R⁴, —COR⁴, —SOR⁴ or —SO₂R⁴, where n is an integer from 0 to 12, or phenyl, heterocyclyl having from 3 to 6 ring atoms, phenyl-(C₁-C₄)-alkyl or heterocyclyl-(C₁-C₄)-alkyl, where each of the 4 latter radicals, on the ring, is unsubstituted or substituted by one or more radicals from the group of halogen, (C₁-C₄)-alkyl, (C₁-C₄)-alkoxy, (C₁-C₄)-haloalkyl and (C₁-C₄)-haloalkoxy, and heterocyclyl is in each case a heterocyclic radical having from 1 to 3 heteroatoms from the group of N, O and S.

In particular,
R¹ is hydrogen, (C₁-C₆)-alkyl, (C₂-C₆)-alkenyl, (C₂-C₆)-alkynyl, (C₃-C₆)-cycloalkyl, (C₁-C₄)-alkyl-(C₃-C₆)-cycloalkyl or a radical of the formula —CO₂R⁴, —(CH₂)ₙCO₂R⁴, —COR⁴, —SOR⁴ or —SO₂R⁴, where n is an integer from 0 to 12, or phenyl.

Very particularly, R¹ is hydrogen or (C₁-C₄)-alkyl.

Preferably,
R² is hydrogen, (C₁-C₁₂)-alkyl, (C₂-C₁₂)-alkenyl, (C₂-C₁₂)-alkynyl, (C₃-C₆)-cycloalkyl, (C₁-C₄)-alkyl-(C₃-C₆)-cycloalkyl or a radical of the formula —CO₂R⁴, —(CH₂)ₙCO₂R⁴, —COR⁴, —SOR⁴ or —SO₂R⁴, where n is an integer from 0 to 12, or optionally substituted aryl, preferably phenyl, which is unsubstituted or substituted by one or more radicals from the group of halogen, (C₁-C₆)-alkyl, (C₁-C₆)-alkoxy, (C₁-C₆)-haloalkyl and (C₁-C₆)-haloalkoxy.

In particular,
R² is hydrogen, (C₁-C₆)-alkyl, (C₂-C₆)-alkenyl, (C₂-C₆)-alkynyl, (C₃-C₆)-cycloalkyl, (C₁-C₄)-alkyl-(C₃-C₆)-cycloalkyl or a radical of the formula —CO₂R⁴, —(CH₂)ₙCO₂R⁴, —COR⁴, —SOR⁴ or —SO₂R⁴, where n is an integer from 0 to 12, or phenyl.

Very particularly, R² is hydrogen or (C₁-C₄)-alkyl.

Preferably,
R³ is (C₁-C₁₂)-alkyl, (C₂-C₁₂)-alkenyl, (C₂-C₁₂)-alkynyl, (C₃-C₆)-cycloalkyl, (C₁-C₄)-alkyl-(C₃-C₆)-cycloalkyl, phenyl, heterocyclyl, phenyl-(C₁-C₄)-alkyl or heterocyclyl-(C₁-C₄)-alkyl, where each of the 4 latter radicals, on the ring, is unsubstituted or substituted by one or more radicals from the group of halogen, (C₁-C₄)-alkyl, (C₁-C₄)-alkoxy, (C₁-C₄)-haloalkyl and (C₁-C₄)-haloalkoxy, and heterocyclyl is in each case a heterocyclic radical having 1 to 3 heteroatoms from the group of N, O and S.

In particular,
R³ is (C₁-C₆)-alkyl, (C₂-C₆)-alkenyl, (C₂-C₆)-alkynyl, (C₃-C₆)-cycloalkyl or (C₁-C₄)-alkyl-(C₃-C₆)-cycloalkyl.

Very particularly, R³ is (C₁-C₄)-alkyl.

Preferably,
R⁴ is (C₁-C₁₂)-alkyl, (C₂-C₁₂)-alkenyl, (C₂-C₁₂)-alkynyl, (C₃-C₆)-cycloalkyl, (C₁-C₄)-alkyl-(C₃-C₆)-cycloalkyl, phenyl, heterocyclyl, phenyl-(C₁-C₄)-alkyl or heterocyclyl-(C₁-C₄)-alkyl, where each of the 4 latter radicals, on the ring, is unsubstituted or substituted by one or more radicals from the group of halogen, (C₁-C₄)-alkyl, (C₁-C₄)-alkoxy, (C₁-C₄)-haloalkyl and (C₁-C₄)-haloalkoxy, and heterocyclyl is in each case a heterocyclic radical having from 1 to 3 heteroatoms from the group of N, O and S.

In particular,
R⁴ is (C₁-C₆)-alkyl, (C₂-C₆)-alkenyl, (C₂-C₆)-alkynyl, (C₃-C₆)-cycloalkyl or (C₁-C₄)-alkyl-(C₃-C₆)-cycloalkyl.

Very particularly, R⁴ is (C₁-C₄)-alkyl.

Preference is also given to processes with compounds in which two or more of the abovementioned preferred features are combined.

Particular preference is given to the processes with enantioselective stages up to the preparation of optically active compounds (IV).

In the preferred embodiment, the fluorinated esters are preparable rapidly and without ecologically difficult secondary components. The reagents used lead, by virtue of their relatively low molecular weight, to small mass flows. The process according to the invention thus constitutes an addition to the prior art, since it allows a very advantageous preparation of α-fluorinated esters from readily obtainable α-hydroxy esters. It was not expected from the known processes that the inventive preparation of compounds of the formula (IV) succeeds efficiently and with the high yield and purity, especially in the case of preparation of chiral non-racemic compounds. This is also true owing to the differences in the reactivities, which are known to be often very large between fluorine compounds compared to chlorine and bromine compounds.

The process for preparing the compounds (IV) consists of the three stages (a1)+(b)+(c) in one variant (see scheme 4), and of the two stages (a2)+(c) in another variant (see scheme 5).

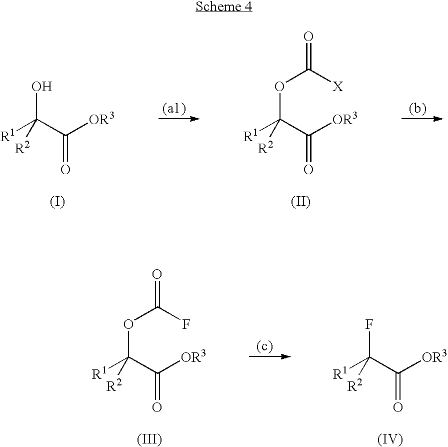

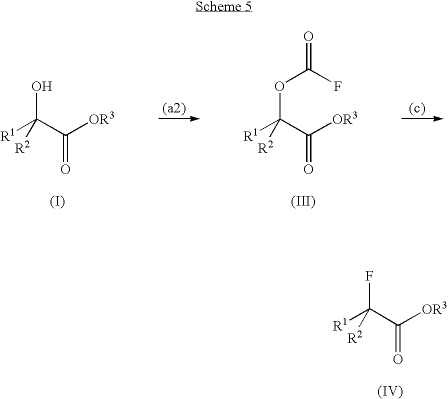

The invention also provides the combination of process stages (b)+(c) or process (c), in which case the compounds (II) or (III) used can then be prepared by another route.

The invention also provides the individual stages (a2) and (b), the compounds of the formula (III) and the combinations of (a1)+(b) and (a2)+(b).

Process stage (a), variants (a1) and (a2):

The hydroxycarboxylic esters of the formula (I) are largely known or can be prepared analogously to known processes (see, for example, EP-A-0163435 and literature cited there).

Their conversion to chloroformates of the formula (II) (X═Cl) according to variant (a1) has already been described in part (see also EP-A-0163435). The chloro- or bromoformates of the formula (II) can be prepared by reacting the compounds (I) with a dihalocarbonyl compound or an equivalent thereof, and the dihalogen compound or its equivalent may, for example, be phosgene (Cl—CO—Cl), carbonyl dibromide (Br—CO—Br), carbonyl bromide chloride (Cl—CO—Br), diphosgene, triphosgene, etc.

Some of the compounds of the formula (II) (especially when X=chlorine) are also known from DE-A-3102516 and can alternatively be prepared by the processes specified there.

The fluoroformates (III) and their preparation according to variant (a2) are novel. In contrast to variant (a1), fluorinated dihalogen compounds or equivalents thereof are used in variant (a2), for example carbonyl difluoride (F—CO—F), carbonyl fluoride chloride (F—CO—Cl), carbonyl fluoride bromide (F—CO—Br), etc.

Variant (a2) can otherwise be performed under the process conditions as known analogously for variant (a1).

Preference is also given to the versions of variant (a1) and (a2) with conversion of optically active compounds of the formula (I) to optically active compounds of the formula (II) or (III). Process stage (a) can thus be performed enantioselectively, which is significant and advantageous for the preparation of optically active compounds (IV) by the overall process.

### Process Stage (b):

The invention reaction of the haloformates of the formula (II) with a fluorinating agent is performed generally at temperatures between −15° C. and 150° C., preferably between −10° C. and 100° C., more preferably between 0° C. and 50° C.

The fluorinating agents used may be customary fluorinating reagents, preferably salt-type fluorine compounds, for example hydrogen fluoride (HF) or mixtures or salts of hydrogen fluoride with organic bases, such as organic amine bases, for example pyridine/hydrogen fluoride, triethylamine/HF or tributylamine/HF. Correspondingly suitable are alkali metal fluorides such as sodium fluoride, potassium fluoride, ammonium fluoride, ammonium or phosphonium fluorides substituted by organic radicals, preferably quaternary ammonium or phosphosium fluorides, for example tetrabutylammonium fluoride or tetraphenylphosphonium fluoride. Preference is given to using pyridine/hydrogen fluoride, triethylamine/HF, NaF or KF.

To prepare the compounds of the fluoroformates of the formula (III), the fluorinating reagent is used in stage (b) preferably in an equimolar amount or in excess. Appropriately, generally between 1.0 and 10 molar equivalents, preferably between 1.0 and 1.6 molar equivalents, of fluorinating reagent are used per mole of reactant of the formula (II), especially a stoichiometric excess of fluorinating reagent. One molar equivalent is understood to mean one mole of a fluorinating reagent which transfers 1 mol of fluorine atoms per mole of reagent. Correspondingly, 1 molar equivalent also means a half mole of a fluorinating reagent which transfers two fluorine atoms per mole of reagent (for example an alkaline earth metal fluoride).

The reaction in step (b) can be performed with or without solvent. The solvents used in the reaction are preferably inert solvents, for example alkylated aromatics, halogenated aromatics, halogenated alkanes, N,N-dialkylated amides, alkylated pyrrolidones, ethers, nitriles, pyridines and sulfolane. Particular preference is given to chlorobenzene, dichlorobenzene, trichlorobenzene, methylene chloride, chloroform, sulfolane, dimethylacetamide, dimethylformamide, acetonitrile, benzonitrile and pyridine. Very particular preference is given to methylene chloride, pyridine, sulfolane and dimethylacetamide.

Process stage (b) can, like all other processes according to the invention, appropriately be performed under standard pressure. However, it is also possible to work under elevated or reduced pressure—preferably between 0.1 bar and 10 bar. Optionally, it is possible in stage (b) to add a catalyst which specifically catalyzes the exchange of the halogen X═Cl or Br in the compound (II) for fluorine and at the same time preferably increases the nucleophilic reactivity of the fluoride anion in the reaction medium. Suitable catalysts are, for example, crown ethers (e.g. 18-[C]-6), polyethylene glycol dialkyl ethers, quaternary ammonium fluorides or quaternary phosphonium fluorides or CsF.

### Process Stage (c):

The inventive reaction (decarboxylation) of the fluoroformates of the formula (III) to the α-fluorinated esters (IV) is generally performed at temperatures between −15° C. and 200° C., preferably between 60° C. and 200° C., more preferably 90° C. and 180° C.

The decarboxylation can be effected without further additives or preferably in the presence of a decarboxylation reagent or catalyst, optionally in the presence of a fluoride source, for example potassium fluoride or hydrogen fluoride (also referred to here together as “catalyst”).

The decarboxylation reagents or catalysts may, for example, be alkali metal halides (for example KF, CsF, or mixtures thereof), aromatic or heteroaromatic tertiary amines and pyridines, for example N,N-dimethylaminopyridine, or phase transfer catalysts or mixtures thereof.

The phase transfer catalysts include, for example:

### (A) quaternary phosphonium or ammonium compounds of the formula (V)

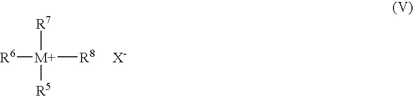 [0093] in which
R⁵, R⁶, R⁷ and R⁸ are each independently C₁-C₂₂-alkyl, in each case optionally substituted aryl or (C₁-C₄-alkyl)aryl, where aryl is defined as phenyl or naphthyl, and said substituents are halogen, C₁-C₄-alkyl, C₁-C₄-alkoxy, nitro or cyano,
X⁻ is one equivalent of a nucleophilic anion (e.g. Cl⁻, Br⁻, I⁻),
M⁺ is N or P,
   or

### (B) amidophosphonium salts of the formula (VI)

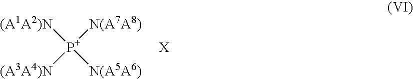 [0098] in which
A¹, A², A³, A⁴, A⁵, A⁶, A⁷ and A⁸ are each independently C₁-C₁₂-alkyl or C₂-C₁₂-alkenyl, C₄-C₈-cycloalkyl, C₆-C₁₂-aryl, C₇-C₁₂-aralkyl, or
A¹A², A³A⁴, A⁵A⁶ and A⁷A⁸ are each independently joined to one another directly or via O or N-A⁹ to give a 3- to 7-membered ring,
A⁹ is C₁-C₄-alkyl,
X⁻ is a nucleophilic anion (e.g. Cl⁻, Br⁻, I⁻), or

### (C) compounds of the formula (VII)

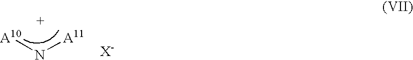 [0104] in which
A¹⁰ and A¹¹ are each independently one of the following radicals

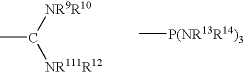

R⁹, R¹⁰, R¹¹, R¹², R¹³ and R¹⁴ are each independently C₁-C₁₀-alkyl, C₂-C₁₀-alkenyl or C₆-C₁₂-aryl, or
R⁹R¹⁰, R¹¹R¹², R¹³R¹⁴ in pairs are each independently joined directly to one another by the nitrogen atoms which are joined in each case to give a 3- to 5-membered, saturated or unsaturated ring which contains one nitrogen atom and otherwise carbon atoms,
   where the radical

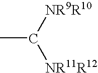

may also be a saturated or unsaturated, 4- to 8-membered ring which contains two nitrogen atoms and otherwise carbon atoms,
X⁻ is one equivalent of a nucleophilic anion (e.g. Cl⁻, Br⁻, I⁻),
   or

### (D) hexaalkylguanidinium salts of the formula (VIII)

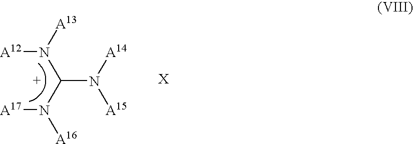 [0112] in which
A¹², A¹³, A¹⁴, A¹⁵, A¹⁶ and A¹⁷ are each independently C₁-C₁₂-alkyl or C₂-C₁₂-alkenyl, C₄-C₈-cycloalkyl, C₆-C₁₂-aryl, C₇-C₁₂-aralkyl, or
A¹²A¹³, A¹⁴A¹⁵ and A¹⁶A¹⁷ are each independently joined to one another directly or via O or N-A¹⁸ to give a 3- to 7-membered ring,
A¹⁸ is C₁-C₄-alkyl,
X⁻ is one equivalent of a nucleophilic anion.

Preference is given to using N,N-dimethylaminopyrimidine (DMAP), CsF, tetraalkylammonium salts, tetraarylphosphonium salts and the hexaalkylguanidinium salts; particular preference is given to CsF, tetraalkylammonium chlorides and tetraalkylammonium bromides.

The compounds mentioned are known to those skilled in the art as phase transfer catalysts.

Optionally, it is possible to add a further catalyst, for example a crown ether (e.g. 18-[C]-6) or a polyethylene glycol dialkyl ether.

Preference is given to the enantioselective procedure of stage (c) using decarboxylation reagents or catalysts. The distinction of reagents and catalysts is only advisable with regard to the different amounts which are optimal in this context; what is common to both substances is that they promote (“catalyze”) the decarboxylation. Suitable decarboxylation reagents or catalysts for the enantioselective procedure are alkali metal halides, preferably fluorides such as KF, CsF or mixtures of fluorides, or aromatic or heteroaromatic tertiary amines such as N-substituted aminopyridines, for example N,N-dimethylaminopyridine, or the phase transfer catalysts mentioned or mixtures thereof.

To prepare the compounds of the formula (IV), the decarboxylation reagent or the decarboxylation catalyst is used generally in a ratio between 0.005 mol and 6 mol, preferably between 0.005 mol and 2 mol, more preferably in a ratio between 0.01 mol and 2 mol, per mole of the compound of the formula (III).

The decarboxylation reaction can be performed with or without solvent. The solvents used in the reaction are preferably solvents from the group of the halogenated aromatics, halogenated alkanes, N,N-dialkylated amides, N-alkylated pyrrolidones, ethers, pyridines, esters, nitriles and sulfolane. Particular preference is given to chlorobenzene, dichlorobenzene, trichlorobenzene, sulfolane and dimethylacetamide. Very particular preference is given to chlorobenzene, sulfolane, products of the reaction itself and dimethylacetamide.

Preference is also given to the reaction without solvent, in which case the reactants and products of the reaction themselves serve as solubilizers or solvents. All processes according to the invention may appropriately be performed under standard pressure. However, it is also possible to work under elevated or reduced pressure—generally between 0.1 bar and 10 bar.

In the working examples which follow, the quantitative data (including percentages) are based on the weight, unless specifically defined otherwise.

The invention is further described by the following non-limiting examples which further illustrate the invention, and are not intended, nor should they be interpreted to, limit the scope of the invention.

### EXAMPLES 1 TO 5

In each case 1.0 g of methyl 2S-[(fluorocarbonyl)oxy]propanoate were converted to methyl 2(R)-fluoropropionate under the conditions recorded in table 1. The temperature listed reports the mean bath temperature of the heating medium. The reactions were performed without distilling out the product. The conversions to the target product are recorded in table 1.

The methyl 2(R)-fluoropropionate product was also characterized by its NMR spectrum:

¹H NMR (400 MHz, CD₃CN): δ=1.51 (dd, 3H, H—C3, J₁=23.9 Hz, J₂=6.8 Hz), 3.73 (s, 3H, ester methyl), 5.06 (dq, 1H, 48.3 Hz, J₂=6.9 Hz).

The enantiomeric excess was determined by gas chromatography on a chiral carrier phase.

TABLE 1

| | | Cat. | KF | | | t | | Conversion | ee* |
|---|---|---|---|---|---|---|---|---|---|
| No. | Cat. | [eq] | [eq.] | T [° C.] | Conc. [%] | [h] | Sol. | [%] | [%] |
| 1 | DMAP | 0.1 | / | 90 | 20 | 6 | MCB | 33 | 95 |
| 2 | DMAP | 0.1 | / | 90 | / | 6 | / | 48 | 88 |
| 3 | DMAP | 0.1 | / | 90 | 20 | 6 | product | 83 | 95 |
| 4 | CsF | 0.1 | / | 160 | 20 | 6 | DMAA | 42 | 96 |
| 5 | 18[C]6 | 0.05 | 2 | 120 | 20 | 6 | DMAA | 63 | 73 |

### Abbreviations for Table 1:

ee=Enantiomeric excess
*=Enantiomeric excess at the end of the reaction time;
Cat.=Decarboxylation catalyst
Cat [eq]=Amount of cat. in molar equivalents based on reactant
T=Reaction temperature (mean temperature of the heating medium)
Conc.=Concentration of reactant at the start of the reaction
Sol.=Solvent
Conversion=Conversion of the reaction based on reactant
DMAP=N,N-dimethylaminopyridine
MCB=Monochlorobenzene
DMAA=Dimethylacetamide
18[C]6=Crown ether 18-crown-6

### EXAMPLES 6-10

In each case 1.0 g of racemic methyl 2-[(fluorocarbonyl)oxy]propanoate was converted to the racemic methyl 2-fluoropropionate under the conditions recorded in table 2. The temperature listed reports the mean bath temperature of the heating medium. The reactions were performed without distilling out the product. The conversions to the target product are recorded in table 2. The methyl 2(R)-fluoropropionate product was also characterized by its NMR spectrum:

¹H NMR (400 MHz, CD₃CN): δ=1.51 (dd, 3H, H—C3, J₁=23.9 Hz, J₂=6.8 Hz), 3.73 (s, 3H, ester methyl), 5.06 (dq, 1H, 48.3 Hz, J₂=6.9 Hz).

TABLE 2

| | | | KF | T | Conc. | t | | |
|---|---|---|---|---|---|---|---|---|
| No | Cat. | [eq] | [eq.] | [° C.] | [%] | [h] | Sol. | Conversion |
| 6 | HBGCl | 0.1 | / | 90 | 40 | 6 | DMAA | 83 |
| 7 | HBGCl | 0.1 | / | rfx. | 40 | 6 | THF | 83 |
| 8 | Bu₄NBr | 0.1 | / | 90 | 20 | 6 | DMAA | 91 |
| 9 | Bu₄NBr | 0.1 | / | 90 | 20 | 6 | xylene | 91 |
| 10 | Bu₄NCl | 0.1 | / | 90 | 20 | 6 | MCB | 83 |

### Abbreviations for Table 2: see Abbreviations for Table 1

### Further Abbreviations for Table 2:

HBGCl=hexabutylguanidinium chloride,
Bu₄NBr=tetrabutylammonium bromide,
Bu₄NCl=tetrabutylammonium chloride,
THF=tetrahydrofuran

### EXAMPLE 11

4.7 g of pyridine were initially charged in 10 ml of dichloromethane and admixed at room temperature with 1.9 g of pyridine-HF complex (M=99.11 g/mol). 10 g of methyl 2-[(chlorocarbonyl)oxy]propanoate (ee. 99%) were added dropwise to this mixture and the resulting reaction mixture was stirred at room temperature overnight. Subsequently, the mixture was added to semiconcentrated hydrochloric acid, the organic phase was removed and the aqueous phase was reextracted with dichloromethane. The combined organic phases were dried (Na₂SO₄) and concentrated. This afforded 7.8 g of methyl 2-[(fluorocarbonyl)oxy]propanoate with a content of 89% (yield: 77%, ee: 99%). The analyses of the enantiomeric excess were undertaken by means of chiral GC.

¹H NMR (400 MHz, CD₃CN): δ=1.55 (dd, 3H, H—C3, J₁=7.1 Hz, J₂=1.6 Hz), 3.76 (s, 3H, ester methyl), 5.12 (dq, 1H, J₁=7.6 Hz, J₂=1.1 Hz).

### EXAMPLE 12

11.2 g of 2-RS-ethylhexyl 2′S-[(chlorocarbonyl)oxy]propanoate were added dropwise at room temperature to a mixture of 3.7 g of potassium fluoride and 0.56 g of the crown ether 18-crown-6 in 25 g of methylene chloride. After stirring overnight, 9.2 g of 2-RS-ethylhexyl 2′S-[(fluorocarbonyl)oxy]propanoate were obtained with a content of 82% (yield: 72% of theory).

¹³C NMR (151 MHz, CD₃CN): δ=11.2 (CH₃CH₂—), 14.3 (C6), 16.9 (C3′), 23.6 (C5), 24.3, 24.4 (CH₃CH₂—), 29.5 (C4), 30.9 (C3), 39.5 (C2), 68.7 (C1), 76.2 (C2′), 145.5 (COF, J=1.9 Hz), 169.9 (C1′).

Having thus described in detail various embodiments of the present invention, it is to be understood that the invention defined by the above paragraphs is not to be limited to particular details set forth in the above description as many apparent variations thereof are possible without departing from the spirit or scope of the present invention.

What is claimed is:

1. A process for preparing compounds of the formula (IV), optionally in optically active form, 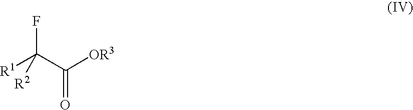
in which
R¹ is hydrogen, (C₁-C₂₄)-alkyl, (C₂-C₂₄)-alkenyl, (C₂-C₂₄)-alkynyl, (C₃-C₉)-cycloalkyl, (C₁-C₆)-alkyl-(C₃-C₉)-cycloalkyl or a radical of the formula —CO₂R⁴, —(CH₂)ₙCO₂R⁴, —COR⁴, —SOR⁴ or —SO₂R⁴, where n is an integer from 0 to 12, or aryl, heterocyclyl, phenyl-(C₁-C₆)-alkyl or heterocyclyl-(C₁-C₆)-alkyl, where each of the 4 latter radicals, on the ring, is unsubstituted or substituted by one or more radicals from the group of halogen, (C₁-C₁₂)-alkyl, (C₁-C₁₂)-alkoxy, (C₁-C₁₂)-haloalkyl and (C₁-C₁₂)-haloalkoxy, and heterocyclyl is in each case a heterocyclic radical having 1 to 3 heteroatoms from the group of N, O and S,
R² is hydrogen, (C₁-C₂₄)-alkyl, (C₂-C₂₄)-alkenyl, (C₂-C₂₄)-alkynyl, (C₃-C₉)-cycloalkyl, (C₁-C₆)-alkyl-(C₃-C₉)-cycloalkyl or a radical of the formula —CO₂R⁴, —(CH₂)ₙCO₂R⁴, —COR⁴, —SOR⁴ or —SO₂R⁴, where n is an integer from 0 to 12, or optionally substituted aryl, preferably phenyl, which is unsubstituted or substituted by one or more radicals from the group of halogen, (C₁-C₁₂)-alkyl, (C₁-C₁₂)-alkoxy, (C₁-C₁₂)-haloalkyl and (C₁-C₁₂)-haloalkoxy,
R³ is (C₁-C₂₄)-alkyl, (C₂-C₂₄)-alkenyl, (C₂-C₂₄)-alkynyl, (C₃-C₉)-cycloalkyl, (C₁-C₆)-alkyl-(C₃-C₉)-cycloalkyl, aryl, heterocyclyl, phenyl-(C₁-C₆)-alkyl or heterocyclyl-(C₁-C₆)-alkyl, where each of the 4 latter radicals, on the ring, is unsubstituted or substituted by one or more radicals from the group of halogen, (C₁-C₁₂)-alkyl, (C₁-C₁₂)-alkoxy, (C₁-C₁₂)-haloalkyl and (C₁-C₁₂)-haloalkoxy, and heterocyclyl is in each case a heterocyclic radical having 1 to 3 heteroatoms from the group of N, O and S,
R⁴ is (C₁-C₂₄)-alkyl, (C₂-C₂₄)-alkenyl, (C₂-C₂₄)-alkynyl, (C₃-C₉)-cycloalkyl, (C₁-C₆)-alkyl-(C₃-C₉)-cycloalkyl, aryl, heterocyclyl, phenyl-(C₁-C₆)-alkyl or heterocyclyl-(C₁-C₆)-alkyl, where each of the 4 latter radicals, on the ring, is unsubstituted or substituted by one or more radicals from the group of halogen, (C₁-C₁₂)-alkyl, (C₁-C₁₂)-alkoxy, (C₁-C₁₂)-haloalkyl and (C₁-C₁₂)-haloalkoxy, and heterocyclyl is in each case a heterocyclic radical having 1 to 3 heteroatoms from the group of N, O and S,
which comprises
(a) reacting a compound of the formula (I) 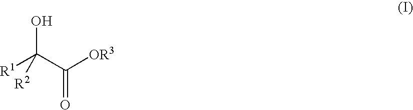
in which R¹, R² and R³ are each as defined in formula (IV)
with a dihalocarbonyl compound or an equivalent thereof to give the compound of the formula (II) [variant (a1)] or (III) [variant (a2)] 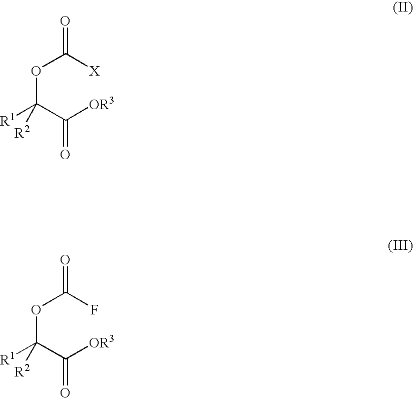
where, in the formulae (II) and (III),
R¹, R² and R³ are each as defined in formula (IV) and
X is a halogen atom from the group of Cl or Br,
and
(b) in the case that the compound (II) has been obtained in stage (a) according to variant (a1), reacting the compound (II) with a fluorinating reagent to give the compound of the formula (III) mentioned,
(c) reacting the compound of the formula (III) obtained in stage (a) or (b) thermally, optionally in the presence of a catalyst, with decarboxylation to give the compound of the formula (IV) mentioned.

2. The process as claimed in claim 1, wherein, in the formula (IV),
R¹ is hydrogen, (C₁-C₁₂)-alkyl, (C₂-C₁₂)-alkenyl, (C₂-C₁₂)-alkynyl, (C₃-C₆)-cycloalkyl, (C₁-C₄)-alkyl-(C₃-C₆)-cycloalkyl or a radical of the formula —CO₂R⁴, —(CH₂)ₙCO₂R⁴, —COR⁴, —SOR⁴ or —SO₂R⁴, where n is an integer from 0 to 12, or phenyl, heterocyclyl having from 3 to 6 ring atoms, phenyl-(C₁-C₄)-alkyl or heterocyclyl-(C₁-C₄)-alkyl, where each of the 4 latter radicals, on the ring, is unsubstituted or substituted by one or more radicals from the group of halogen, (C₁-C₄)-alkyl, (C₁-C₄)-alkoxy, (C₁-C₄)-haloalkyl and (C₁-C₄)-haloalkoxy, and heterocyclyl is in each case a heterocyclic radical having from 1 to 3 heteroatoms from the group of N, O and S,
R² is hydrogen, (C₁-C₁₂)-alkyl, (C₂-C₁₂)-alkenyl, (C₂-C₁₂)-alkynyl, (C₃-C₆)-cycloalkyl, (C₁-C₄)-alkyl-(C₃-C₆)-cycloalkyl or a radical of the formula —CO₂R⁴, —(CH₂)ₙCO₂R⁴, —COR⁴, —SOR⁴ or —SO₂R⁴, where n is an integer from 0 to 12, or optionally substituted aryl, preferably phenyl, which is unsubstituted or substituted by one or more radicals from the group of halogen, (C₁-C₆)-alkyl, (C₁-C₆)-alkoxy, (C₁-C₆)-haloalkyl and (C₁-C₆)-haloalkoxy,
R³ is (C₁-C₁₂)-alkyl, (C₂-C₁₂)-alkenyl, (C₂-C₁₂)-alkynyl, (C₃-C₆)-cycloalkyl, (C₁-C₄)-alkyl-(C₃-C₆)-cycloalkyl, phenyl, heterocyclyl, phenyl-(C₁-C₄)-alkyl or heterocyclyl-(C₁-C₄)-alkyl, where each of the 4 latter radicals, on the ring, is unsubstituted or substituted by one or more radicals from the group of halogen, (C₁-C₄)-alkyl, (C₁-C₄)-alkoxy, (C₁-C₄)-haloalkyl and (C₁-C₄)-haloalkoxy, and heterocyclyl is in each case a heterocyclic radical having from 1 to 3 heteroatoms from the group of N, O and S, and
R⁴ is (C₁-C₁₂)-alkyl, (C₂-C₁₂)-alkenyl, (C₂-C₁₂)-alkynyl, (C₃-C₆)-cycloalkyl, (C₁-C₄)-alkyl-(C₃-C₆)-cycloalkyl, phenyl, heterocyclyl, phenyl-(C₁-C₄)-alkyl or heterocyclyl-(C₁-C₄)-alkyl, where each of the 4 latter radicals, on the ring, is unsubstituted or substituted by one or more radicals from the group of halogen, (C₁-C₄)-alkyl, (C₁-C₄)-alkoxy, (C₁-C₄)-haloalkyl and (C₁-C₄)-haloalkoxy, and heterocyclyl is in each case a heterocyclic radical having from 1 to 3 heteroatoms from the group of N, O and S.

3. The process as claimed in claim 1, wherein, in the formula (IV),
R¹ is hydrogen, (C₁-C₆)-alkyl, (C₂-C₆)-alkenyl, (C₂-C₆)-alkynyl, (C₃-C₆)-cycloalkyl, (C₁-C₄)-alkyl-(C₃-C₆)-cycloalkyl or a radical of the formula —CO₂R⁴, —(CH₂)ₙCO₂R⁴, —COR⁴, —SOR⁴ or —SO₂R⁴, where n is an integer from 0 to 12, or phenyl,
R² is hydrogen, (C₁-C₆)-alkyl, (C₂-C₆)-alkenyl, (C₂-C₆)-alkynyl, (C₃-C₆)-cycloalkyl, (C₁-C₄)-alkyl-(C₃-C₆)-cycloalkyl or a radical of the formula —CO₂R⁴, —(CH₂)ₙCO₂R⁴, —COR⁴, —SOR⁴ or —SO₂R⁴, where n is an integer from 0 to 12, or phenyl,
R³ is (C₁-C₆)-alkyl, (C₂-C₆)-alkenyl, (C₂-C₆)-alkynyl, (C₃-C₆)-cycloalkyl or (C₁-C₄)-alkyl-(C₃-C₆)-cycloalkyl and
R⁴ is (C₁-C₆)-alkyl, (C₂-C₆)-alkenyl, (C₂-C₆)-alkynyl, (C₃-C₆)-cycloalkyl or (C₁-C₄)-alkyl-(C₃-C₆)-cycloalkyl.

4. The process as claimed in claim 1, wherein an aromatic or heteroaromatic tertiary amine, a phase transfer catalyst and/or a fluoride source is used for the thermal decarboxylation in stage (c).

5. The process as claimed in claim 1, wherein the decarboxylation in stage (c) is performed at temperatures between 60 and 200° C.

6. The process as claimed in claim 1, wherein the reactions are performed using a chiral, non-racemic reactant, and the decarboxylation in stage (c) is conducted enantioselectively in the presence of a decarboxylation reagent or catalyst from the group of DMAP, KF, CsF, tetraalkylammonium chlorides, tetralkylphosphonium chlorides, hexaalkylguanidinium chlorides, hexaalkylguanidinium fluorides and mixtures of the compounds from the aforementioned group.

7. The process as claimed in claim 1, wherein the solvent in stage (c) is a halogenated aromatic, an N,N-dialkylated amide, sulfolane or an ester.

8. The process as claimed in claim 1, wherein from 0.005 to 6 mol of decarboxylation reagent/catalyst are used in stage (c) per mole of compound of the formula (III).

9. A process for preparing compounds of the formula (IV), optionally in optically active form, 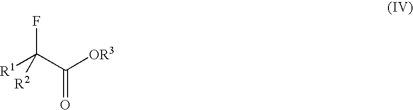
in which
R¹ is hydrogen, (C₁-C₂₄)-alkyl, (C₂-C₂₄)-alkenyl, (C₂-C₂₄)-alkynyl, (C₃-C₉)-cycloalkyl, (C₁-C₆)-alkyl-(C₃-C₉)-cycloalkyl or a radical of the formula —CO₂R⁴, —(CH₂)ₙCO₂R⁴, —COR⁴, —SOR⁴ or —SO₂R⁴, where n is an integer from 0 to 12, or aryl, heterocyclyl, phenyl-(C₁-C₆)-alkyl or heterocyclyl-(C₁-C₆)-alkyl, where each of the 4 latter radicals, on the ring, is unsubstituted or substituted by one or more radicals from the group of halogen, (C₁-C₁₂)-alkyl, (C₁-C₁₂)-alkoxy, (C₁-C₁₂)-haloalkyl and (C₁-C₁₂)-haloalkoxy, and heterocyclyl is in each case a heterocyclic radical having 1 to 3 heteroatoms from the group of N, O and S,
R² is hydrogen, (C₁-C₂₄)-alkyl, (C₂-C₂₄)-alkenyl, (C₂-C₂₄)-alkynyl, (C₃-C₉)-cycloalkyl, (C₁-C₆)-alkyl-(C₃-C₉)-cycloalkyl or a radical of the formula —CO₂R⁴, —(CH₂)ₙCO₂R⁴, —COR⁴, —SOR⁴ or —SO₂R⁴, where n is an integer from 0 to 12, or optionally substituted aryl, preferably phenyl, which is unsubstituted or substituted by one or more radicals from the group of halogen, (C₁-C₁₂)-alkyl, (C₁-C₁₂)-alkoxy, (C₁-C₁₂)-haloalkyl and (C₁-C₁₂)-haloalkoxy,
R³ is (C₁-C₂₄)-alkyl, (C₂-C₂₄)-alkenyl, (C₂-C₂₄)-alkynyl, (C₃-C₉)-cycloalkyl, (C₁-C₆)-alkyl-(C₃-C₉)-cycloalkyl, aryl, heterocyclyl, phenyl-(C₁-C₆)-alkyl or heterocyclyl-(C₁-C₆)-alkyl, where each of the 4 latter radicals, on the ring, is unsubstituted or substituted by one or more radicals from the group of halogen, (C₁-C₁₂)-alkyl, (C₁-C₁₂)-alkoxy, (C₁-C₁₂)-haloalkyl and (C₁-C₁₂)-haloalkoxy, and heterocyclyl is in each case a heterocyclic radical having 1 to 3 heteroatoms from the group of N, O and S,
R⁴ is (C₁-C₂₄)-alkyl, (C₂-C₂₄)-alkenyl, (C₂-C₂₄)-alkynyl, (C₃-C₉)-cycloalkyl, (C₁-C₆)-alkyl-(C₃-C₉)-cycloalkyl, aryl, heterocyclyl, phenyl-(C₁-C₆)-alkyl or heterocyclyl-(C₁-C₆)-alkyl, where each of the 4 latter radicals, on the ring, is unsubstituted or substituted by one or more radicals from the group of halogen, (C₁-C₁₂)-alkyl, (C₁-C₁₂)-alkoxy, (C₁-C₁₂)-haloalkyl and (C₁-C₁₂)-haloalkoxy, and heterocyclyl is in each case a heterocyclic radical having 1 to 3 heteroatoms from the group of N, O and S,
which comprises
(c) reacting a compound of the formula (III) 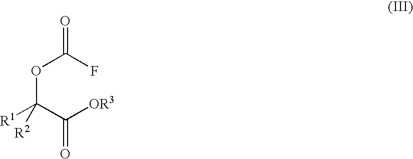
where, in formula (III), R¹, R² and R³ are each as defined in formula (IV), thermally, optionally in the presence of a catalyst, with decarboxylation to give the compound of the formula (IV) mentioned.

10. A process for preparing compounds of the formula (IV), optionally in optically active form, which comprises reacting a compound of the formula (II) 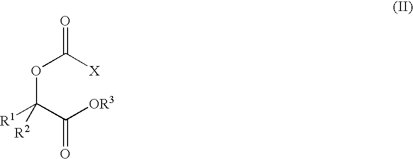
in which R¹, R² and R³ are each as defined in formula (IV) according to claim 1 and X is an halogen atom from the group of Cl and Br,
with a fluorinating agent to give the compound of the formula (III) 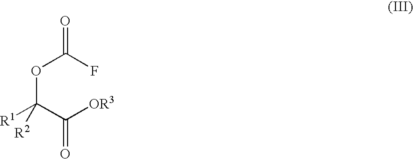
where, in formula (III), R¹, R² and R³ are each as defined in the formula (IV) mentioned,
and reacting the resulting compound (III) thermally, optionally in the presence of a catalyst, with decarboxylation to give the compound of the formula (IV) mentioned.

11. A compound of the formula (III), optionally in optically active form, as defined in claim 9.

12. A compound of the formula (III) as claimed in claim 11, wherein
R¹ is hydrogen, (C₁-C₆)-alkyl, (C₂-C₆)-alkenyl, (C₂-C₆)-alkynyl, (C₃-C₆)-cycloalkyl, (C₁-C₄)-alkyl-(C₃-C₆)-cycloalkyl or a radical of the formula —CO₂R⁴, —(CH₂)ₙCO₂R⁴, —COR⁴, —SOR⁴ or —SO₂R⁴, where n is an integer from 0 to 12, or phenyl,
R² is hydrogen, (C₁-C₆)-alkyl, (C₂-C₆)-alkenyl, (C₂-C₆)-alkynyl, (C₃-C₆)-cycloalkyl, (C₁-C₄)-alkyl-(C₃-C₆)-cycloalkyl or a radical of the formula —CO₂R⁴, —(CH₂)ₙCO₂R⁴—COR⁴, —SOR⁴ or —SO₂R⁴, where n is an integer from 0 to 12, or phenyl,
R³ is (C₁-C₆)-alkyl, (C₂-C₆)-alkenyl, (C₂-C₆)-alkynyl, (C₃-C₆)-cycloalkyl or (C₁-C₄)-alkyl-(C₃-C₆)-cycloalkyl and
R⁴ is (C₁-C₆)-alkyl, (C₂-C₆)-alkenyl, (C₂-C₆)-alkynyl, (C₃-C₆)-cycloalkyl or (C₁-C₄)-alkyl-(C₃-C₆)-cycloalkyl.

13. The compound of claim 12, wherein the compound is chiral and non-racemic.
